(19) United States
(12) Patent Application Publication
(10) Pub. No.: US 2018/0070850 A1
(43) Pub. Date: Mar. 15, 2018

(54) APPARATUS AND METHOD FOR DETECTING BODY COMPOSITION AND CORRELATING IT WITH COGNITIVE EFFICIENCY

(71) Applicants: Karen S. Stafford, Gilbert, AZ (US); Shiladitya Laskar, Bangalore (IN); Ramesh Pendakur, Gaston, OR (US)

(72) Inventors: Karen S. Stafford, Gilbert, AZ (US); Shiladitya Laskar, Bangalore (IN); Ramesh Pendakur, Gaston, OR (US)

(21) Appl. No.: 15/266,783

(22) Filed: Sep. 15, 2016

(51) Int. Cl. A61B 5/053; A61B 5/00

(52) U.S. Cl. CPC A61B 5/0537; A61B 5/4875; A61B 5/6803; A61B 5/486; A61B 5/16; A61B 5/0022; A61B 5/6825; A61B 5/6897; A61B 5/7465

(57) ABSTRACT

An apparatus and method is provided for detecting body composition and for correlating body composition with cognitive efficiency. The apparatus comprises: at least two sensors to sense a potential difference of a human body region; a computing device electrically coupled to the at least two sensors, wherein the computing device is to: detect a body hydration level according to the sensed potential difference; and notify a user if the body hydration level reaches a predetermined threshold.

### BACKGROUND

Most individuals are unaware of their chronic poor hydration habits. They may be unaware of a link between low personal hydration and cognitive performance, which can have serious physiological impact(s). The human body continuously loses water through breathing, sweating, and urinating. Body hydration levels can impact cognitive efficiency of a person. Mental processing efficiency goes down when fluid homeostasis is disturbed.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIG. 1 illustrates an ensemble of wearable devices including a device with one or more bio-impedance sensors (e.g., on handle gripping regions) used for detecting body composition, according to some embodiments of the disclosure.

FIG. 2 illustrates bio-impedance sensors at the palm rest area of a laptop, according to some embodiments of the disclosure.

FIG. 3 illustrates bio-impedance sensors at the gripping region of a gaming device, according to some embodiments of the disclosure.

FIG. 4 illustrates bio-impedance sensors at the holding area on the back of a tablet.

FIG. 5 illustrates bio-impedance sensors in the cuff of a shirt, according to some embodiments of the disclosure.

FIG. 6 illustrates a three-dimensional view of a bio-impedance sensor, according to some embodiments of the disclosure.

FIG. 7 describes a working model of an embodiment.

FIG. 8 illustrates a flow diagram which explains how dehydration affects cognition.

FIG. 9 illustrates a flowchart of a method of various embodiments.

FIG. 10 illustrates a process according to various embodiments of the disclosure.

FIG. 11 illustrates an electrode system with a machine readable storage medium (or media) having instructions for bio-impedance analysis, according to some embodiments of the disclosure.

FIG. 12 illustrates a smart device or a computer system or a SoC (System-on-Chip) for processing data collected by one of more sensors for bio-impedance analysis, according to some embodiments.

### DETAILED DESCRIPTION

Mild dehydration can lead to lower cognitive efficiency which is expected to affect psychomotor activities and decision making abilities in an individual. Various embodiments described here can detect body hydration levels and assess cognitive impact as well. The system of various embodiments can be very important for the users who work for long hours in hot environments or in those work places which requires intelligent decisions to reduce the chances of occupational hazards such as aviation.

Cognitive performance is adversely affected due to dehydration which is consistent among all age groups. Loosing 2% of body water can impair performance in tasks which require attention, hand-eye coordination, and immediate memory skills. Various embodiments described here can be used with several form-factors and can opportunistically determine body composition and cognitive efficiency via bio-impedance sensors. Some embodiments describe an apparatus and method for bioelectrical impedance analysis. Bioelectrical impedance analysis can be used for body fat analysis. In some embodiments, total body water can be determined with integrated sensors in different form factors. In some embodiments, a very low level current at different frequencies is applied and body impedance or resistance is measured. In some embodiments, the impedance measurement is compared to known user anthropometric attributes and optimum hydration levels to determine total body water and cognitive efficiency.

In some embodiments, total body water and cognitive efficiency are opportunistically sensed and interpreted at several points during a user's day, keeping circadian rhythm into consideration. In some embodiments, patterns of hydration are determined to encourage users to maintain their hydration optimally and enhance their cognitive abilities such as decision making (e.g., a big presentation coming up). In some embodiments, an application is provided which takes the data from both user's hydration level and digital calendar to alert the user to consume a required amount of water to satisfy the deficit.

Some embodiments relate to a method and system for accurately measuring body fat, body hydration levels, and nutritional status while simultaneously assessing user mood and cognitive efficiency. Some embodiments measure body hydration level and simultaneously predict cognitive efficiency. The embodiments described here can interpret opportunistic bio-impedance measurement into relevant physiological information and can predict user cognitive attributes by using minimal input data.

Some embodiments relate to a system which deduces cognitive abilities and alertness levels by detecting body hydration levels and can advise a user to take actions upon it. Some embodiments can detect hydration status and other physiological parameters and relate then to cognitive efficiency of an individual in real time. Some embodiments can detect hydration levels with higher accuracy with minimal anthropometric inputs. In some embodiments, body fat percentage which seems to fluctuate with varied degree of hydration is taken care of by appropriate usage of various predictive equations which is applicable to users of various age groups across ethnic origins.

Some embodiments described here have an excellent productivity application since the system is capable of evaluating cognitive efficiency along with other parameters such as body fat percentage, body hydration levels, nutritional status, and fat percentage. The apparatus of various embodiments can also be used as a complete healthcare manager which can guide its user to enhance his or her performance at work and outside as well. Some embodiments target a wider array of user base from all age groups.

In the following description, numerous details are discussed to provide a more thorough explanation of embodiments of the present disclosure. It will be apparent, however, to one skilled in the art, that embodiments of the present disclosure may be practiced without these specific details. In other instances, well-known structures and devices are shown in block diagram form, rather than in detail, in order to avoid obscuring embodiments of the present disclosure.

Note that in the corresponding drawings of the embodiments, signals are represented with lines. Some lines may be thicker, to indicate more constituent signal paths, and/or have arrows at one or more ends, to indicate primary information flow direction. Such indications are not intended to be limiting. Rather, the lines are used in connection with one or more exemplary embodiments to facilitate easier understanding of a circuit or a logical unit. Any represented signal, as dictated by design needs or preferences, may actually comprise one or more signals that may travel in either direction and may be implemented with any suitable type of signal scheme.

Throughout the specification, and in the claims, the term “connected” means a direct connection, such as electrical, mechanical, or magnetic connection between the things that are connected, without any intermediary devices. The term “coupled” means a direct or indirect connection, such as a direct electrical, mechanical, or magnetic connection between the things that are connected or an indirect connection, through one or more passive or active intermediary devices. The term “circuit” or “module” may refer to one or more passive and/or active components that are arranged to cooperate with one another to provide a desired function. The term “signal” may refer to at least one current signal, voltage signal, magnetic signal, or data/clock signal. The meaning of “a,” “an,” and “the” include plural references. The meaning of “in” includes “in” and “on.”

The term “scaling” generally refers to converting a design (schematic and layout) from one process technology to another process technology and subsequently being reduced in layout area. The term “scaling” generally also refers to downsizing layout and devices within the same technology node. The term “scaling” may also refer to adjusting (e.g., slowing down or speeding up—i.e. scaling down, or scaling up respectively) of a signal frequency relative to another parameter, for example, power supply level. The terms “substantially,” “close,” “approximately,” “near,” and “about,” generally refer to being within +/−10% of a target value.

Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner.

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C). The term s “left,” “right,” “front,” “back,” “top,” “bottom” “over,” “under,” the like in the description and in the claims, if any, are used for descriptive purposes and not necessarily for describing permanent relative positions.

For purposes of the embodiments, the transistors in various circuits, sensors, and logic blocks described here are metal oxide semiconductor (MOS) transistors or their derivatives, where the MOS transistors include drain, source, gate, and bulk terminals. The transistors and/or the MOS transistor derivatives also include Tri-Gate and FinFET transistors, Gate All Around Cylindrical Transistors, Tunneling FET (TFET), Square Wire, or Rectangular Ribbon Transistors, or other devices implementing transistor functionality like carbon nanotubes or spintronic devices. MOSFET symmetrical source and drain terminals i.e., are identical terminals and are interchangeably used here. A TFET device, on the other hand, has asymmetric Source and Drain terminals. Those skilled in the art will appreciate that other transistors, for example, Bi-polar junction transistors—BJT PNP/NPN, BiCMOS, CMOS, etc., may be used without departing from the scope of the disclosure.

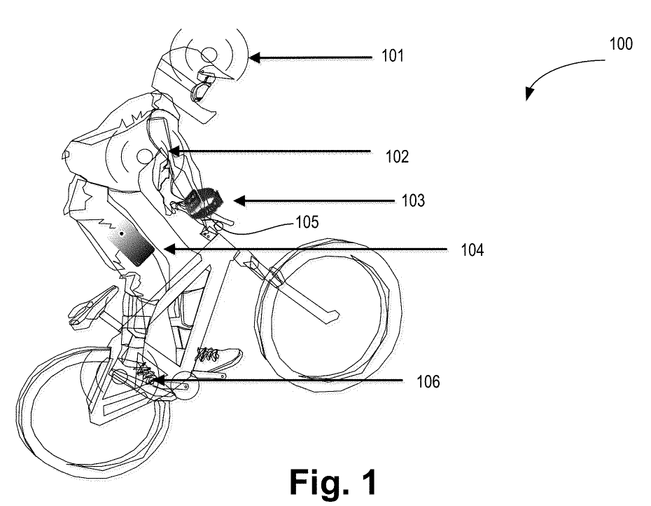

FIG. 1 illustrates ensemble 100 of wearable devices including a device with one or more sensors used for detecting body composition and correlating it with cognitive efficiency, according to some embodiments of the disclosure. In this example, ensemble 100 is on a person and his/her ride (here, a bicycle). However, the embodiments are not limited to such. Other scenarios of wearable devices and their usage may work with the various embodiments. For example, sensors can be embedded in the googles worn by the person such that the sensors are in direct contact with the body of the person.

In some embodiments, the sensor node(s) are part of a wearable device. The term “wearable device” (or wearable computing device) generally refers to a device coupled to a person. For example, devices or accessories (such as sensors, cameras, speakers, microphones (mic), smartphones, smart watches, hair bands, hats, undergarments, helmet, hair pins, pairs of spectacles, hair brush, comb, etc.) which are directly attached on a person, on the person's clothing, or on a person's accessories are within the scope of wearable devices.

In some examples, wearable computing devices may be powered by a main power supply such as an AC/DC (Alternating Current and/or Direct Current) power outlet. In some examples, wearable computing devices may be powered by a battery. In some examples, wearable computing devices may be powered by a specialized external source based on Near Field Communication (NFC). The specialized external source may provide an electromagnetic field that may be harvested by circuitry at the wearable computing device. Another way to power the wearable computing device is electromagnetic field associated with wireless communication, for example, WLAN transmissions. WLAN transmissions use far field radio communications that have a far greater range to power a wearable computing device than NFC transmission. WLAN transmissions are commonly used for wireless communications with most types of terminal computing devices.

For instance, the WLAN transmissions may be used in accordance with one or more WLAN standards based on Carrier Sense Multiple Access with Collision Detection (CSMA/CD) such as those promulgated by the Institute of Electrical Engineers (IEEE). These WLAN standards may be based on CSMA/CD wireless technologies such as Wi-Fi™ and may include Ethernet wireless standards (including progenies and variants) associated with the IEEE 802.11-2012 Standard for Information technology—Telecommunications and information exchange between systems—Local and metropolitan area networks—Specific requirements Part 11: WLAN Media Access Controller (MAC) and Physical Layer (PHY) Specifications, published March 2012, and/or later versions of this standard (“IEEE 802.11”).

In some embodiments, ensemble 100 of wearable devices includes device 101 (e.g., camera, microphone, etc.) mounted on a helmet, device 102 (e.g., blood pressure sensor, body composition detector etc.) strapped on the person's arm, device 103 (e.g., a smart watch that can function as a terminal controller or a device to be controlled), device 104 (e.g., a smart phone and/or tablet in a pocket of the person's clothing), device 105 attached on the handle grips such that they are in direct contact with the hands of the rider, and device 106 (e.g., an accelerometer to measure paddling speed). In some embodiments, ensemble 100 of wearable devices has the capability to communicate by wireless energy harvesting mechanisms or other types of wireless transmission mechanisms.

In some embodiments, the helmet includes an inner covering that directly couples to a person's head. In some embodiments, the inner covering includes processing logic, body composition sensors, analog-to-digital converter (ADC), temperature sensor, bus, micro-controller or processor, antenna, and battery pack. In some embodiments, when the helmet is taken off and placed on its stand (not shown), the battery pack of inner covering charges.

In some embodiments, the one or more sensors of inner covering sense different parameters of the person's body. In some embodiments, the signals for the sensors of inner covering are digitized and transmitted to a terminal device (e.g., cloud, personal computer, laptop, etc.) over Wi-Fi (or other wireless technologies) by a micro-controller. In some embodiments, software (or machine executable instructions) in the terminal device are used to quantify the digitized data and correlate it with cognitive efficiency using bio-impedance analysis.

In some embodiments, device 102 includes bio-impedance sensors that are part of an electrode in contact with a person's arm. In some embodiments, the bio-impedance sensors detect a body hydration level according to a sensed potential difference in the person's arm. In some embodiments, a wireless interface in device 102 is used to notify a user or a computing device if the body hydration level reaches a predetermined threshold.

In some embodiments, the bio-impedance sensors inject a first current of a first frequency into the arm. In some embodiments, the bio-impedance sensors inject a second current of a second frequency into the arm, where the first frequency is higher than the second frequency. In some embodiments, a processor or logic may be part of device 102, and this processor and logic may measure a first reactance using the first current of the first frequency and also measure a first resistance using the first current of the first frequency. In some embodiments, the processor or logic measures a second reactance using the second current of the second frequency, and measures a second resistance using the second current of the first frequency. In some embodiments, the processor or logic determines a phase angle according to the measured first reactance, first resistance, second reactance, and second resistance. In some embodiments, the processor or logic determines an indicator of human wellness according to the determined phase angle and anthropometric parameters. In some embodiments, the post processing (e.g., measuring first reactance, first resistance, second reactance, and second resistance) of the sensed data (e.g., potential difference) is done by a terminal device or a server away from device 102.

In some embodiments, bio-impedance sensors or device 105 are attached to the grips of the bicycle handles to determine the potential difference via hand palms. Like device 102, device 105 may include a processor or logic to measure a first reactance using the first current of the first frequency and also measure a first resistance using the first current of the first frequency. In some embodiments, the processor or logic of device 105 measures a second reactance using the second current of the second frequency, and measures a second resistance using the second current of the first frequency. In some embodiments, the processor or logic of device 105 determines a phase angle according to the measured first reactance, first resistance, second reactance, and second resistance. In some embodiments, the processor or logic of device 105 determines an indicator of human wellness according to the determined phase angle and anthropometric parameters. In some embodiments, the post processing (e.g., measuring first reactance, first resistance, second reactance, and second resistance) of the sensed data (e.g., potential difference) is done by a terminal device or a server away from device 105.

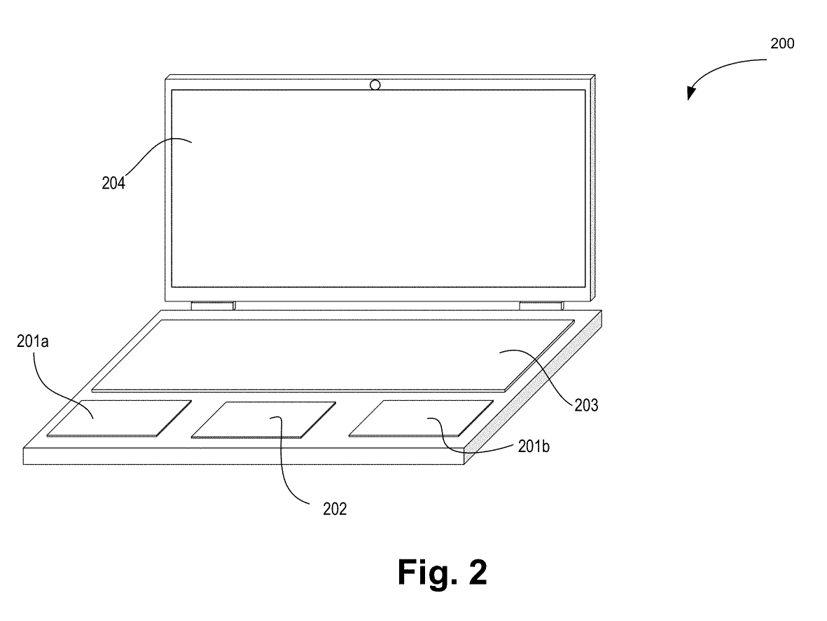

FIG. 2 illustrates bio-impedance sensors at the palm rest area of laptop 200, according to some embodiments of the disclosure. In some embodiments, laptop 200 includes a pair of bio-impedance sensors 201a and 201b on either sides of a mouse pad 202. In some embodiments, bio-impedance sensors 201a and 201b are positioned to so that they can get in direct contact with a user's palms. In some embodiments, the keys of keyboard 203 may also have embedded bio-impedance sensors that can get in direct contact with a user's fingers. In some embodiments, bio-impedance sensors 201a and 201b provide data by wired or wireless means to a processor of laptop 200 for post processing. In some embodiments, raw data, table based data, or any other visual form of data associated with the bio-impedance is projected on screen 204 of laptop 200. For example, indicators of the user's wellness may be displayed on screen 204. As such, a user may see his or her wellness parameters in real-time.

Some embodiments involve instrumenting mainstream computing platforms (e.g. laptop keypads, tablet surfaces, etc.) with embedded sensors that opportunistically sense the user's physiological parameters (such as bio-impedance). Bio-impedance spectroscopy is a consumer preferred methodology which is simple and non-invasive. It can electronically determine and estimate body composition. Body composition has a significant health impact such as lower body hydration levels that can lead to adverse consequences in all age groups.

Bio-impedance estimates body composition in terms of total body water, fat free mass, and fat mass. The various embodiments described here can measure bio-impedance and can interpret them to physiologically relevant information to the user. The bioelectrical impedance analysis estimates body composition using the difference of conductivity which arises due to fluctuations in body water. The adipose tissue constitutes the major proportion of fat present in a body. Conductivity is decreased with increase in body fat percentage. When a weak alternating current (AC) signal flows through the human body, impedance has a steady relationship with body composition. The measured impedance is an indicator of the ratio between conductive and non-conductive tissue.

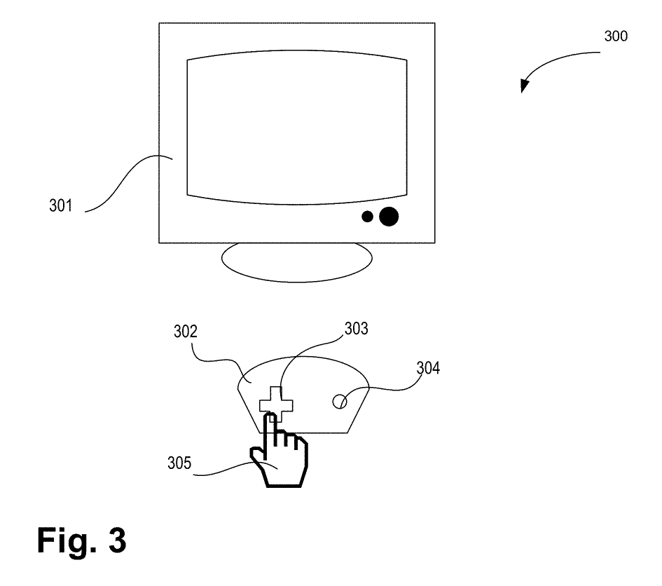

FIG. 3 illustrates bio-impedance sensors at the gripping region of a gaming system 300, according to some embodiments of the disclosure. Gaming system 300 comprises display 301 and gaming controller 302. In some embodiments, the buttons 303 and 304 of the gaming controller 302 have embedded bio-impedance sensors that can get in direct contact with a user's fingers (see hand 305).

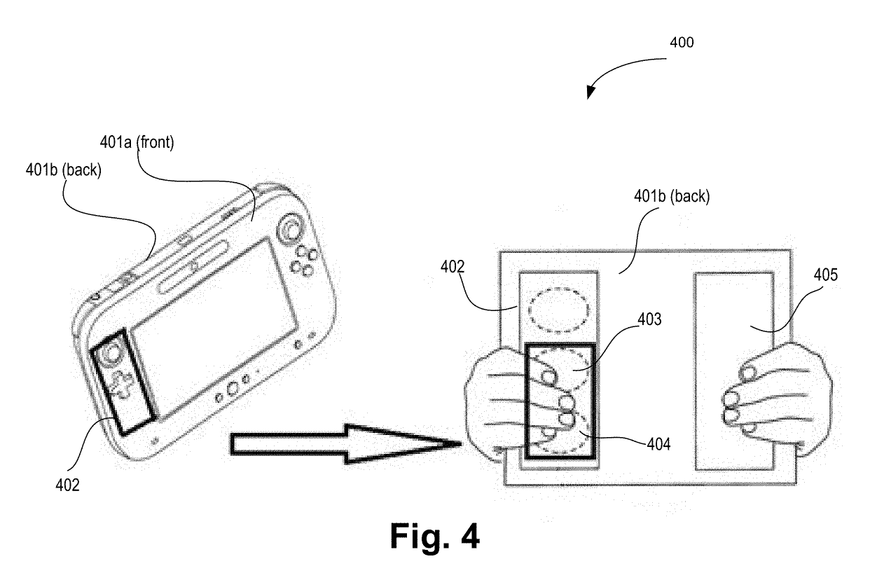

FIG. 4 illustrates apparatus 400 with bio-impedance sensors at the holding area in the back of a tablet. Here, the front region of the tablet is indicated by region 401a while the back side of the table is indicated by region 401b. The front region 401a includes a screen, and a region of control buttons 402. When a user holds the tablet with two hands (e.g., when playing a game), the fingers of the user wrap the tablet and are in direct contact with the back region 401b of the tablet. In some embodiments, regions 402 and 405 are identified in the back region 401b of the tablet. These regions 402 and 405 have embedded bio-impedance sensors, in accordance with some embodiments. Here, three bio-impedance sensors are shown in region 402. However, any number of sensors can be used as technology for sensors results in smaller sized sensors. In this example, sensors 403 and 404 are in direct contact with the figures of the users. These sensors are used to measure bio-impedance, which is then used to determine the wellness of the user.

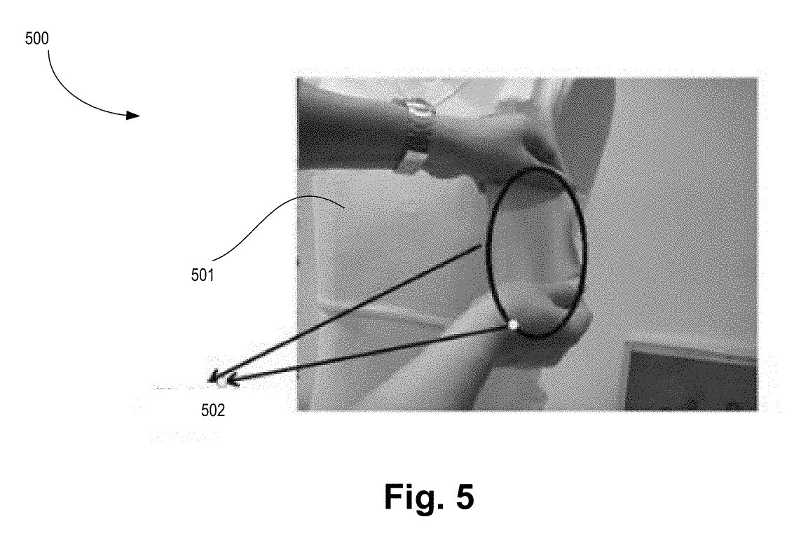

FIG. 5 illustrates a view 500 of bio-impedance sensors 502 in the cuff of shirt 501, according to some embodiments of the disclosure. These sensors 502 are in direct contact with the wrist of a user, and as such can measure bio-impedance of the user.

The form factors described with reference to FIGS. 1-5 are not an exclusive list of form factors that may have bio-impedance sensors. Other form factors that are in direct contact to human palm-rest area or other parts of the body can be used. For example, baseball bat, steering wheels, yoke, etc. may have embedded bio-impedance sensors. These sensors can opportunistically sense impedance and interpret user's physiological and psychological conditions, in accordance with various embodiments.

In some embodiments, the bio-impedance sensors (e.g., sensors on gripping region 105, sensors 201a/b, sensors on game console buttons 303 and 304, sensors 403 and 404 on a back region 401b of a tablet, sensors 502 embedded in a cuff of a shirt 501, etc., may use electrodes with oleoophobic coating. Oleoophobic coating provides a smooth finish that does not leave behind figure prints of users. In some embodiments, the oleoophobic coating is applied over electrical conductors (e.g., a mesh of electrodes). In some embodiments, electrodes are placed in a bi-polar manner. For example, electrodes 201a and 201b are placed on either sides of a mouse pad. In some embodiments, electrodes are placed in a tetra-polar position. Tetra-polar electrodes result in higher accuracy than bi-polar electrodes, in accordance with some embodiments.

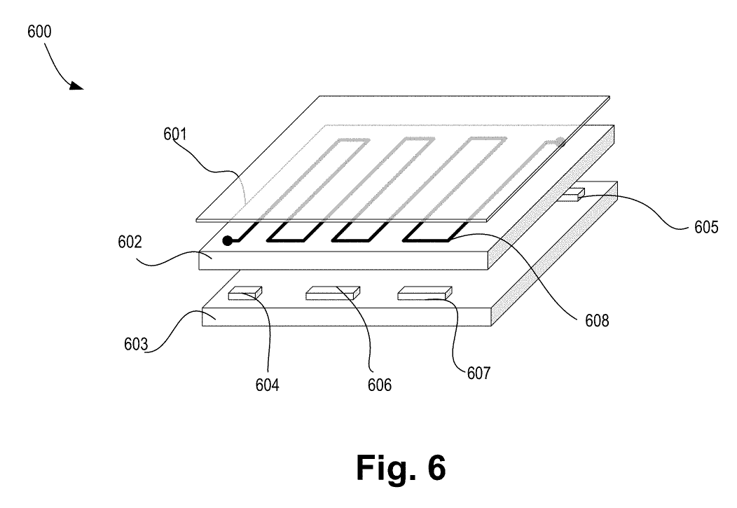

FIG. 6 illustrates a three dimensional (3D) view 600 of a bio-impedance sensor, according to some embodiments of the disclosure. It is pointed out that those elements of FIG. 6 having the same reference numbers (or names) as the elements of any other figure can operate or function in any manner similar to that described, but are not limited to such.

In some embodiments, bio-impedance sensor includes a layer of oleoophobic coating 601, a layer of 602 of conductor mesh, and a flexible printed circuit board (PCB) 603 having one or more active and passive devices. In some embodiments, the back side of PCM 603 has a sticky material to attach the bio-impedance sensor to a device (e.g., laptop, handle of a bicycle, steering wheel, etc.).

In some embodiments, PCB 603 includes contacts 604 and 605, processor or logic 606, and antenna 607. In some embodiments, layer 202 has a conductive mesh 608. In some embodiments, PCM 603 and layer 602 may be combined into a single layer. For example, conductive mesh 608 may be embedded in PCB 603. In some embodiments, contacts 604 and 605 comprise highly conductive metals. In some embodiments, processor or logic 606 is one of: A Digital Signal Processor (DSP), an Application Specific Integrated Circuit (ASCI), a general purpose Central Processing Unit (CPU), or a low power logic implementing a simple finite state machine to perform the various methods described here.

In some embodiments, antenna 607 is an antenna array. In some embodiments, the antenna array may comprise one or more of directional or omnidirectional antennas 1 through ‘N,’ where ‘N’ is an integer, including monopole antennas, dipole antennas, loop antennas, patch antennas, microstrip antennas, coplanar wave antennas, or other types of antennas suitable for transmission of Radio Frequency (RF) signals. In some multiple-input multiple-output (MIMO) embodiments, the antenna array is separated to take advantage of spatial diversity.

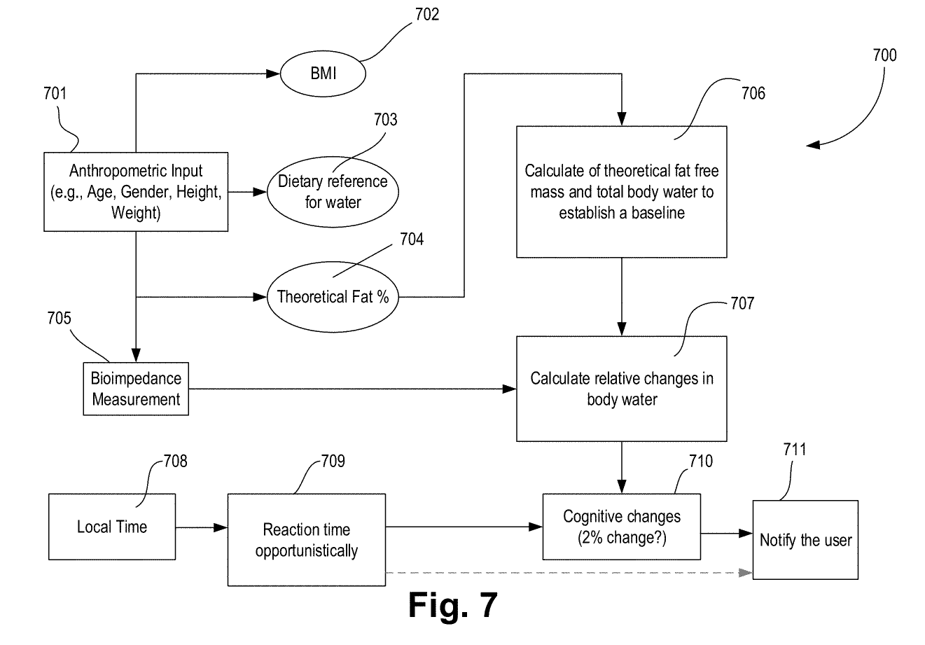

FIG. 7 illustrates flowchart 700 showing a working model of an embodiment. It is pointed out that those elements of FIG. 7 having the same reference numbers (or names) as the elements of any other figure can operate or function in any manner similar to that described, but are not limited to such.

Although the blocks in the flowchart with reference to FIG. 7 are shown in a particular order, the order of the actions can be modified. Thus, the illustrated embodiments can be performed in a different order, and some actions/blocks may be performed in parallel. Some of the blocks and/or operations listed in FIG. 7 are optional in accordance with certain embodiments. The numbering of the blocks presented is for the sake of clarity and is not intended to prescribe an order of operations in which the various blocks must occur. Additionally, operations from the various flows may be utilized in a variety of combinations.

At block 701, a computing device (e.g., a handheld device, server, etc.) receives anthropometric input such as age, gender, height, weight, skin coloration, etc. In some embodiments, the anthropometric input is entered manually for a user. In some embodiments, anthropometric input is captured by a camera which takes a picture of the user and estimates age, gender, height, weight, skin coloration, etc. In some embodiments, a combination of manual entries and automatic estimations (e.g., via a camera or similar device) is used to capture anthropometric information. In some embodiments, additional information like body mass index (BMI) 702, and dietary reference for water 703 is also collected.

In some embodiments, anthropometric information 701, BMI 702 information, and/or dietary reference for water 703 is used to calculate or estimate a theoretical fat percentage (%) 704. Any known method can be used for estimating the theoretical fat %.

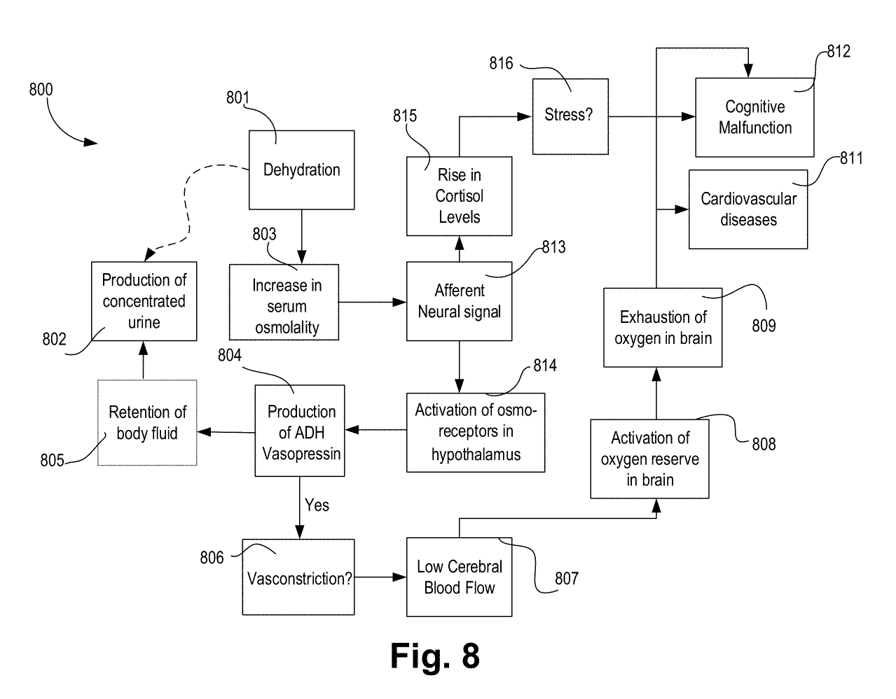

At block 705 bio-impedance sensors determine the potential difference in a body region of a user (e.g., palms of a person). One such bio-impedance measurement process is illustrated with reference to FIG. 8. Referring back to FIG. 7, theoretical fat % from block 704 is used to calculate theoretical fat free mass and total body water to establish a baseline as shown by block 706. Any known method can be used for establishing this baseline. The system of various embodiments utilizes an algorithm which can detect optimal hydration level of a user and can establish a personalized baseline.

In some embodiments, at block 707, relative changes in body water for the user is estimated or calculated using the established baseline from block 706 and the bio-impedance measurement from block 705. In some embodiments, the calculated relative changes in body water is used to determine any cognitive change in the user.

In some embodiments, cognitive changes are determined in view of local time collected at block 708, reaction time collected at block 709, and/or relative changes in body water 707. In some embodiments, at block 711 the system of various embodiments notifies the user about the changes of his hydration level at different time intervals after opportunistic sensing (see dotted line). In some embodiments, information about cognitive efficiency is calculated opportunistically by measuring the reaction times (e.g., block 709) towards a visual stimulus or somatosensory stimulus to the user. Some embodiments periodically measure body hydration levels and other physiological parameters such as body fat percentage using bio-impedance methodology.

In some embodiments, at block 710 cognitive changes are identified when there is predetermined change (e.g., 2% change) in body water. Cognitive performance is adversely affected due to dehydration which is consistent among all age groups. Loosing 2% of body water can impair performance in tasks which require attention, hand-eye coordination and immediate memory skills. At block 711, the user is notified about the cognitive change. For example, the user is alerted on a smart device (e.g., a smart phone), a phone call, a text message, etc. Some embodiments utilize various population specific predictive equations to obtain more accurate readings.

FIG. 8 illustrates flow diagram 800 which explains how dehydration affects cognition. Some embodiments are based on the null hypothesis which states that dehydration impacts cognitive efficiency negatively, but temporarily. Poor hydration levels can have serious chronic impacts on psychomotor activities which can lead to cognitive impairment. Conversely, fluid intake can influence mood and cognition positively. Hence the system exploits the correlation of body composition and its cognitive impact on the body.

As shown in flow diagram 800, dehydration 801 may cause production of concentrated urine 802 and may increase serum osmolality 803. Low fluid intake can lead to dehydration which increases plasma osmolality. The change in plasma osmolality triggers osmo-receptors in the brain. Plasma osmolality triggers an afferent neural signal 813 to the hypothalamus in the brain and stimulates the magnocellular neuro-secretory neurons in the paraventricular nucleus of hypothalamus and supra-optic nucleus (or osmo-receptors) 814 to produce vasopressin (ADH) 804.

Vasopressin is a neuro-peptide secreted by posterior pituitary which is involved in primary functions such as constriction of blood vessels and retention of water 805 in the body leading to production of concentrated urine 802. Vasopressin system 806 is also suspected to get activated under physiological stress such as prolonged dehydration. Vasopressin is an antidiuretic, which constricts blood vessels and also effects cerebral blood flow 807. Cerebral blood flow 807 modulates neural activity and hence cognitive function.

Water is an essential nutrient and promotes osmoregulation to remove toxic wastes from the body. During progressive dehydration, the blood flow to brain decreases and triggers an unsuspecting oxygen reserve of the brain 808. The depletion or exhaustion 809 of oxygen reserves affects the cognitive performance and also leads to production of toxic by-products in brain which can accumulate over time and attenuate neural systems. For example, cardiovascular diseases 811 and/or cognitive malfunction 812 can be caused by the toxic by-products in the brain.

Large water fluxes take place across blood-brain barrier and promote detoxification of the brain with the help of cerebrospinal fluid. Persistent and chronic dehydrated conditions due to delay in onset of thirst in aging users can lead to accumulation of toxic by-products such as beta-amyloid plaques in the brain and can cause permanent cognitive impairment 812. Hence the bio-impedance system of various embodiments can detect body hydration changes and protect user from adverse side-effects of dehydration.

Dehydration 801 increases plasma or serum osmolality 803, which triggers a feedback mechanism to release ADH Vasopressin 804 to retain water and concentrates the urine 802. The increasing cortisol level with onset of dehydration in blood plasma is an indicator of hypothalamic neural activity. The hypothalamus in the brain acts as a command center to various glandular functions in the brain which in turn modulates stress response 816. Hence dehydration also modulates stress levels 816. Blood cortisol levels are indicators of stress in an individual.

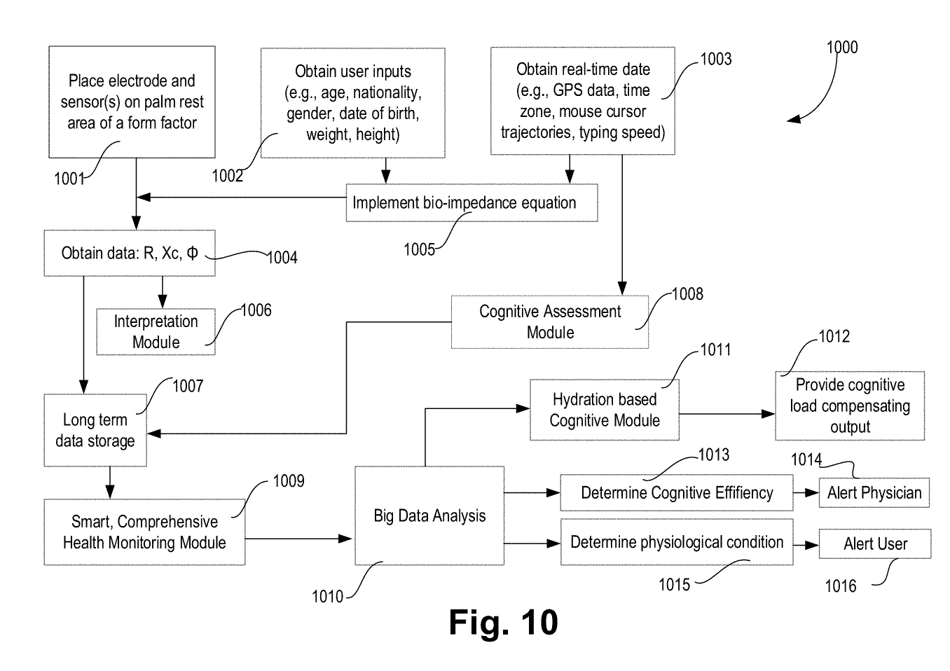

Stress 816 can be a major hurdle to the productivity of a person and can do a long term damage leading to chronic hypertension and other cardio-vascular 811 risks. The bio-impedance sensing described in various embodiments can guide a user to maintain body fluid homeostasis by alerting their optimum hydration requirement and hence maintain cortisol levels 815 in the blood. Some embodiments can be a healthcare manager to the users and can help them manage their quality of life. The mechanism is described in FIG. 10.

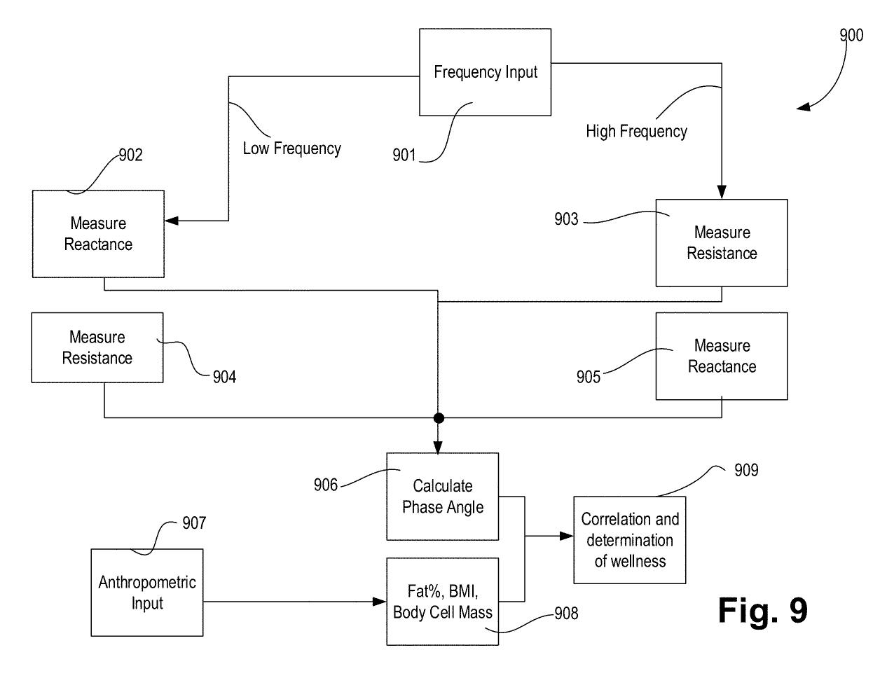

FIG. 9 illustrates flowchart 900 of method of bio-impedance measurement using the bio-impedance sensors, according to some embodiments. Although the blocks in the flowchart with reference to FIG. 9 are shown in a particular order, the order of the actions can be modified. Thus, the illustrated embodiments can be performed in a different order, and some actions/blocks may be performed in parallel. Some of the blocks and/or operations listed in FIG. 9 are optional in accordance with certain embodiments. The numbering of the blocks presented is for the sake of clarity and is not intended to prescribe an order of operations in which the various blocks must occur. Additionally, operations from the various flows may be utilized in a variety of combinations.

In some embodiments, at block 901 bio-impedance sensor injects a first current (e.g., an alternating current (AC)) of first frequency (e.g., low frequency) to a portion of a body of a user. At block 902, first reactance is measured using the received first current through the body. At block 901 bio-impedance sensor injects a second AC current of second frequency (e.g., high frequency) to the portion of the body. The current levels of injected current are generally weak enough to not cause an electric shock to the user but strong enough to determine a potential difference. By injecting currents with different frequencies, phase angle is calculated.

At block 903, second reactance is measured using the received second current through the body. In some embodiments, first reactance is converted to first resistance at block 904. In some embodiments, second reactance is converted to second resistance at block 905.

In some embodiments, bio-impedance phase angle is calculated at block 906 using first and second reactances, and first and second resistances. Phase angle can determine cellular integrity and hence is an indicator of malnutrition. The phase angle parameter may also be used as an independent indicator of health/cellular health in clinical practices. Major shifts in phase angle may determine if there is a need for a check-up or a clinical visit depending upon the user age group.

At block 907, anthropometric input is received by a computing device communicatively coupled to the bio-impedance sensors. At block 908, anthropometric input is used to determine percentage of fat, BMI, and body cell mass. At block 909, the calculated phase angle from block 906 and percentage of fat, BMI, and body cell mass from block 908 are used to correlate determination of wellness. The results can alert the user and help him manage the quality of life. The system can be used by diabetics and can help them mitigate other health impacts on them.

FIG. 10 illustrates a process or flowchart 1000 according to various embodiments of the disclosure. Although the blocks in the flowchart with reference to FIG. 10 are shown in a particular order, the order of the actions can be modified. Thus, the illustrated embodiments can be performed in a different order, and some actions/blocks may be performed in parallel. Some of the blocks and/or operations listed in FIG. 10 are optional in accordance with certain embodiments. The numbering of the blocks presented is for the sake of clarity and is not intended to prescribe an order of operations in which the various blocks must occur. Additionally, operations from the various flows may be utilized in a variety of combinations.

The system is capable of neuropsychological assessment to determine cognitive efficiency of a user. At block 1001, one or more electrodes with bio-impedance sensors are placed on a form factor (e.g., bicycle handle grip, laptop, tablet, etc.). At block 1002, anthropometric input (e.g., age, nationality, gender, date of birth, weight, height, etc.) of a user is collected. At block 1003, real-time environmental data is collected. For example, global positioning system (GPS) data, time zone (e.g., Pacific Time zone), mouse cursor position, mouse cursor trajectories, tying speed, etc. is collected. Data from blocks 1001, 1002, and 1003 may be stored in memory for different users.

At block 1004, bio-impedance sensors determine the reactance, resistance, and phase angles as described with reference to FIG. 9. Referring back to FIG. 10, in some embodiments, reactance, resistance, and phase angles are determined in view of raw data obtained from block 1002. In some embodiments, reactance, resistance, and phase angles are determined in view of real-time data obtained from block 1003.

In some embodiments, at block 1005, a bio-impedance equation is provided to calculate reactance, resistance, and phase angles according to the injected and received currents of different frequencies. By changing the bio-impedance equation, values for reactance, resistance, and phase angles may change. In some embodiments, bio-impedance equation is provided to an interpretation module 1006. In some embodiments, at block 1004 reaction times are obtained by providing a visual or somatosensory stimulus to the user when the bio-impedance sensors are used along with main-stream computing devices.

In some embodiments, at block 1006, the information from blocks 1001, 1002, 1003, 1004, and/or 1005 is provided to an interpretation module. In some embodiments, interpretation module may be a logic of a processor. In some embodiments, interpretation module determines the total body water, fat free mass, fat mass to estimate hydration levels and fat status of the user. In some embodiments, the bio-impedance system can obtain impedance profile and simultaneously advise user on his cognitive efficiency and mood. Reaction time obtained by the system can determine the impact of hydration on cognitive function and is a good estimate of a user's alertness which tends to get adversely affected during poor hydration levels. The bio-impedance system of some embodiments can be helpful in beverage assessment which aims at maintaining the electrolyte levels of the drinker.

In some embodiments, data from blocks 1001, 1002, 1003, 1004 and/or 1005 are stored in large memories (e.g., non-volatile memories) for long term storage 1007 and for big data analysis 1010. In some embodiments, the long term storage 1007 facilitates health status monitoring and cognitive decline monitoring in old and middle aged patients. In some embodiments, data from block 103 is used by a cognitive assessment module 1009 to assess the impact of the parameters from block 103 on cognitive efficiency. The findings of cognitive assessment module 1009 may be stored in long term storage 1007.

In some embodiments, Smart and Comprehensive Health Monitoring Module 1009 is used to perform big data analysis 1010 for detecting neuropsychological performance and body hydration levels opportunistically. In some embodiments, Smart and Comprehensive Health Monitoring Module 1009 can determine acute dehydration and hydration state of a subject and advise user on consumption of water. In some embodiments, Smart and Comprehensive Health Monitoring Module 1009 determines fat levels. These fat levels along with user physical attributes (e.g., data from block 1003) can be used as input for fitness monitoring. For example, the determined fat levels can be used to motivate a subject to implement fitness regime.

The big data analysis at block 1010 can be used for determining phase angle value changes. These phase angle value changes can determine overall physiological condition as indicated by block 1015. Any abnormality (determined by any suitable criteria) can then be used to alert a user as indicated by block 1016. Big data analysis at block 1010 can also assist with determining cognitive efficiency as indicated by block 1013. For example, long term chronic malfunctions such as decline in cognitive efficiency can be detected at block 1013.

The bio-impedance system of various embodiments can prove beneficial to all age groups but particularly aging population which tend to lose fluid homeostasis due to weakened perception of thirst and can cause serious adverse consequences. The bio-impedance system of various embodiments also performs various opportunistic cognitive assessments at block 1013 which can also be used as an early detection system of cognitive impairment in aging population. Any abnormality (determined by any suitable criteria) can then be used to alert a physician as indicated by block 1014.

At block 1011, hydration based cognitive module can detect hydration levels of the person or subject. Dehydration affects eye sight and vision which is a major cause of symptoms like head-ache. The bio-impedance system of various embodiments can detect hydration levels of a person and can help in improving their learning efficiency without straining physically.

At block 1012, the cognitive information is provided to the user to change user behavior. In some embodiments, bio-impedance system can be a plug-in to an array of learning and productivity applications. The bio-impedance system of various embodiments can also be used as an excellent business productivity manager. For example, the interception of low hydration can be completed prior to big calendar events such as important presentations, combined with other sensor information. The bio-impedance system of various embodiments can be used as guidance system to keep a track of oral hydration and hence can aid in enhancing the user's efficiency. The bio-impedance system of various embodiments can be used by workers who work in hot conditions and tend to lose body water faster and can guide towards better physical and mental performance at work place.

The bio-impedance system of various embodiments can be used with variety of form factors such as in the cuffs of a shirt which consists of textile electrode. The bio-signals obtained can be sent to cloud or any communication device for further processing and relevant physiological information can be given to the wearer. The bio-impedance system of various embodiments can particularly assist users such as defense personnel, sportsmen, mountaineers and other users who frequently undergo extreme physical activities in general.

The bio-impedance system of various embodiments can be implemented with various gaming consoles to detect hydration levels of professional gamers and can instruct them on their hydration levels facilitating them to perform executive functions with higher efficiency. The physiological recordings can be incorporated into designing of virtual environments suiting the physiological condition of the user. The bio-impedance system of various embodiments can be integrated in various simulator platforms to assess cognitive impact of a user related to his hydration levels.

The bio-impedance analysis of various embodiments may focus on fluid homeostasis of a person. Fluid homeostasis is important in well-being. Changes in fluid homeostasis is related with kidney disorders, diabetes, and hypertension. But to predict them, patterns may be needed which are possible by the long term data storage.

Large shifts in the long term data can determine possibility of a chronic malfunction, for example. The bio-impedance system of various embodiments can determine if there is a possibility of chronic disorder owing to any large shifts in the long term data. The data collected by the various embodiments may help the application learn and evolve (e.g., via machine learning and/or pattern matching). The phase angle parameter is also used as an independent indicator of health and cellular health in clinical practices.

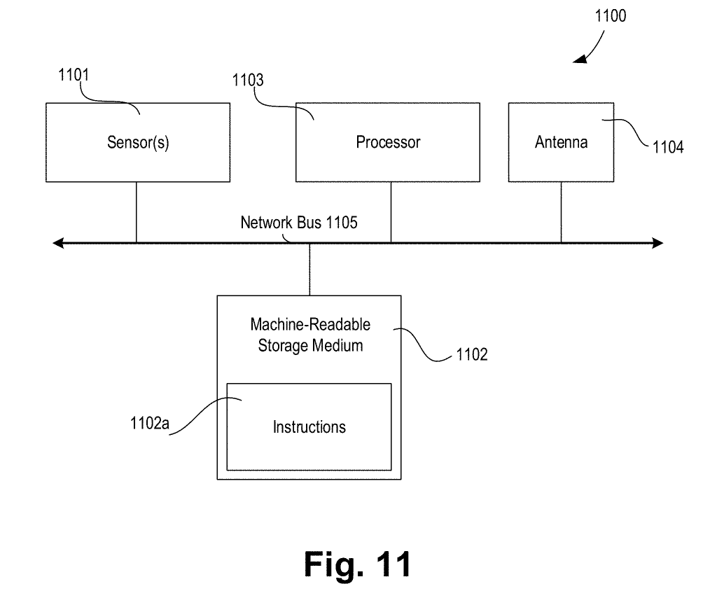

FIG. 11 illustrates an electrode system with a machine readable storage medium (or media) having instructions for bio-impedance analysis, according to some embodiments of the disclosure. It is pointed out that those elements of FIG. 11 having the same reference numbers (or names) as the elements of any other figure can operate or function in any manner similar to that described, but are not limited to such.

In some embodiments, system 1100 comprises one or more sensors 1101, Machine-Readable Storage Medium 1102, low power processor 1103, antenna 1104, and Network Bus 1105. In some embodiments, the one or more sensors 1101 are the bio-impedance sensors described with reference to various embodiments. In some embodiments, processor 1103 is a Digital Signal Processor (DSP), an Application Specific Integrated Circuit (ASIC), a general purpose Central Processing Unit (CPU), or a low power logic implementing a simple finite state machine to perform the method of flowcharts 700, 900, and/or 1000 of various embodiments. Here, Machine-Readable Storage Medium 1102 is also referred to as tangible machine readable medium.

In some embodiments, the various logic blocks of system 1100 are coupled together via Network Bus 1105. Any suitable protocol may be used to implement Network Bus 1105. In some embodiments, Machine-Readable Storage Medium 1102 includes Instructions 1102a (also referred to as the program software code/instructions) for processing signals (current and/or voltages) sensed by the bio-impedance sensors as described with reference to various embodiments and flowchart.

Program software code/instructions 1102a associated with flowcharts 700, 900, and/or 1000 (and/or various embodiments) and executed to implement embodiments of the disclosed subject matter may be implemented as part of an operating system or a specific application, component, program, object, module, routine, or other sequence of instructions or organization of sequences of instructions referred to as “program software code/instructions,” “operating system program software code/instructions,” “application program software code/instructions,” or simply “software” or firmware embedded in processor. In some embodiments, the program software code/instructions associated with flowcharts 700, 900, and/or 1000 (and/or various embodiments) are executed by system 1100.

In some embodiments, the program software code/instructions 1102a associated with flowcharts 700, 900, and/or 1000 are stored in a computer executable storage medium 1102 and executed by Processor 1103. Here, computer executable storage medium 1102 is a tangible machine readable medium that can be used to store program software code/instructions and data that, when executed by a computing device, causes one or more processors (e.g., Processor 1103) to perform a method(s) as may be recited in one or more accompanying claims directed to the disclosed subject matter.

The tangible machine readable medium 1102 may include storage of the executable software program code/instructions 1102a and data in various tangible locations, including for example ROM, volatile RAM, non-volatile memory and/or cache and/or other tangible memory as referenced in the present application. Portions of this program software code/instructions 1102a and/or data may be stored in any one of these storage and memory devices. Further, the program software code/instructions can be obtained from other storage, including, e.g., through centralized servers or peer to peer networks and the like, including the Internet. Different portions of the software program code/instructions and data can be obtained at different times and in different communication sessions or in the same communication session.

The software program code/instructions 1102a (associated with flowcharts 700, 900, and/or 1000 and other embodiments) and data can be obtained in their entirety prior to the execution of a respective software program or application by the computing device. Alternatively, portions of the software program code/instructions 1102a and data can be obtained dynamically, e.g., just in time, when needed for execution. Alternatively, some combination of these ways of obtaining the software program code/instructions 1102a and data may occur, e.g., for different applications, components, programs, objects, modules, routines or other sequences of instructions or organization of sequences of instructions, by way of example. Thus, it is not required that the data and instructions be on a tangible machine readable medium in entirety at a particular instance of time.

Examples of tangible computer-readable media 1102 include but are not limited to recordable and non-recordable type media such as volatile and non-volatile memory devices, read only memory (ROM), random access memory (RAM), flash memory devices, floppy and other removable disks, magnetic storage media, optical storage media (e.g., Compact Disk Read-Only Memory (CD ROMS), Digital Versatile Disks (DVDs), etc.), among others. The software program code/instructions may be temporarily stored in digital tangible communication links while implementing electrical, optical, acoustical or other forms of propagating signals, such as carrier waves, infrared signals, digital signals, etc. through such tangible communication links.

In general, tangible machine readable medium 1102 includes any tangible mechanism that provides (i.e., stores and/or transmits in digital form, e.g., data packets) information in a form accessible by a machine (i.e., a computing device), which may be included, e.g., in a communication device, a computing device, a network device, a personal digital assistant, a manufacturing tool, a mobile communication device, whether or not able to download and run applications and subsidized applications from the communication network, such as the Internet, e.g., an iPhone®, Galaxy®, Blackberry® Droid®, or the like, or any other device including a computing device. In one embodiment, processor-based system is in a form of or included within a PDA (personal digital assistant), a cellular phone, a notebook computer, a tablet, a game console, a set top box, an embedded system, a TV (television), a personal desktop computer, etc. Alternatively, the traditional communication applications and subsidized application(s) may be used in some embodiments of the disclosed subject matter.

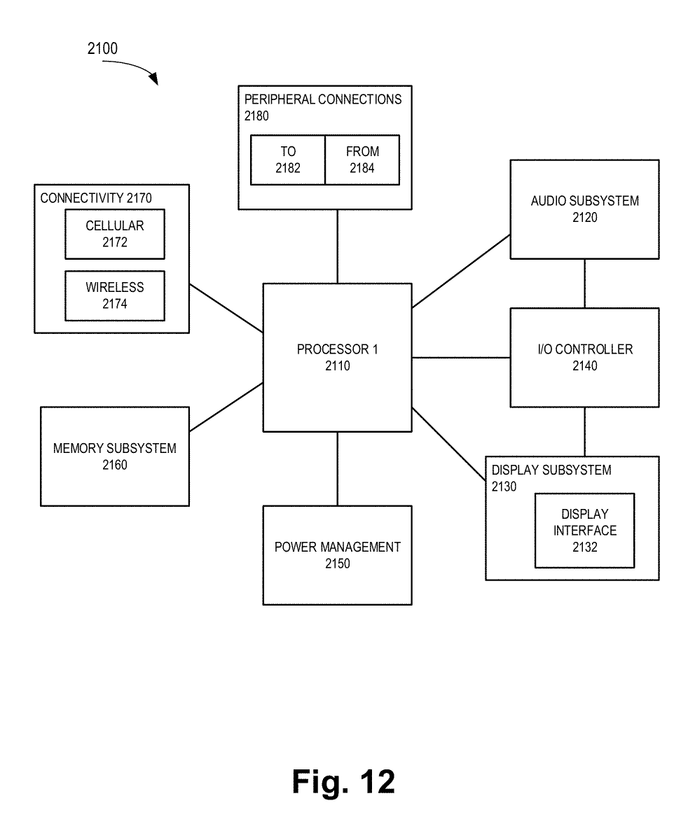

FIG. 12 illustrates a smart device or a computer system or a SoC (System-on-Chip) for processing data collected by one of more sensors for bio-impedance analysis, according to some embodiments. It is pointed out that those elements of FIG. 12 having the same reference numbers (or names) as the elements of any other figure can operate or function in any manner similar to that described, but are not limited to such.

FIG. 12 illustrates a block diagram of an embodiment of a mobile device in which flat surface interface connectors could be used. In some embodiments, computing device 2100 represents a mobile computing device, such as a computing tablet, a mobile phone or smart-phone, a wireless-enabled e-reader, or other wireless mobile device. It will be understood that certain components are shown generally, and not all components of such a device are shown in computing device 2100.

In some embodiments, computing device 2100 includes a first processor 2110. The various embodiments of the present disclosure may also comprise a network interface within 2170 such as a wireless interface so that a system embodiment may be incorporated into a wireless device, for example, cell phone or personal digital assistant.

In one embodiment, processor 2110 can include one or more physical devices, such as microprocessors, application processors, microcontrollers, programmable logic devices, or other processing means. The processing operations performed by processor 2110 include the execution of an operating platform or operating system on which applications and/or device functions are executed. The processing operations include operations related to I/O (input/output) with a human user or with other devices, operations related to power management, and/or operations related to connecting the computing device 2100 to another device. The processing operations may also include operations related to audio I/O and/or display I/O.

In one embodiment, computing device 2100 includes audio subsystem 2120, which represents hardware (e.g., audio hardware and audio circuits) and software (e.g., drivers, codecs) components associated with providing audio functions to the computing device. Audio functions can include speaker and/or headphone output, as well as microphone input. Devices for such functions can be integrated into computing device 2100, or connected to the computing device 2100. In one embodiment, a user interacts with the computing device 2100 by providing audio commands that are received and processed by processor 2110.

Display subsystem 2130 represents hardware (e.g., display devices) and software (e.g., drivers) components that provide a visual and/or tactile display for a user to interact with the computing device 2100. Display subsystem 2130 includes display interface 2132, which includes the particular screen or hardware device used to provide a display to a user. In one embodiment, display interface 2132 includes logic separate from processor 2110 to perform at least some processing related to the display. In one embodiment, display subsystem 2130 includes a touch screen (or touch pad) device that provides both output and input to a user.

I/O controller 2140 represents hardware devices and software components related to interaction with a user. I/O controller 2140 is operable to manage hardware that is part of audio subsystem 2120 and/or display subsystem 2130. Additionally, I/O controller 2140 illustrates a connection point for additional devices that connect to computing device 2100 through which a user might interact with the system. For example, devices that can be attached to the computing device 2100 might include microphone devices, speaker or stereo systems, video systems or other display devices, keyboard or keypad devices, or other I/O devices for use with specific applications such as card readers or other devices.

As mentioned above, I/O controller 2140 can interact with audio subsystem 2120 and/or display subsystem 2130. For example, input through a microphone or other audio device can provide input or commands for one or more applications or functions of the computing device 2100. Additionally, audio output can be provided instead of, or in addition to display output. In another example, if display subsystem 2130 includes a touch screen, the display device also acts as an input device, which can be at least partially managed by I/O controller 2140. There can also be additional buttons or switches on the computing device 2100 to provide I/O functions managed by I/O controller 2140.

In one embodiment, I/O controller 2140 manages devices such as accelerometers, cameras, light sensors or other environmental sensors, or other hardware that can be included in the computing device 2100. The input can be part of direct user interaction, as well as providing environmental input to the system to influence its operations (such as filtering for noise, adjusting displays for brightness detection, applying a flash for a camera, or other features).

In one embodiment, computing device 2100 includes power management 2150 that manages battery power usage, charging of the battery, and features related to power saving operation. Memory subsystem 2160 includes memory devices for storing information in computing device 2100. Memory can include nonvolatile (state does not change if power to the memory device is interrupted) and/or volatile (state is indeterminate if power to the memory device is interrupted) memory devices. Memory subsystem 2160 can store application data, user data, music, photos, documents, or other data, as well as system data (whether long-term or temporary) related to the execution of the applications and functions of the computing device 2100.

Elements of embodiments are also provided as a machine-readable medium (e.g., memory 2160) for storing the computer-executable instructions. The machine-readable medium (e.g., memory 2160) may include, but is not limited to, flash memory, optical disks, CD-ROMs, DVD ROMs, RAMs, EPROMs, EEPROMs, magnetic or optical cards, phase change memory (PCM), or other types of machine-readable media suitable for storing electronic or computer-executable instructions. For example, embodiments of the disclosure may be downloaded as a computer program (e.g., BIOS) which may be transferred from a remote computer (e.g., a server) to a requesting computer (e.g., a client) by way of data signals via a communication link (e.g., a modem or network connection).

Connectivity 2170 includes hardware devices (e.g., wireless and/or wired connectors and communication hardware) and software components (e.g., drivers, protocol stacks) to enable the computing device 2100 to communicate with external devices. The computing device 2100 could be separate devices, such as other computing devices, wireless access points or base stations, as well as peripherals such as headsets, printers, or other devices.

Connectivity 2170 can include multiple different types of connectivity. To generalize, the computing device 2100 is illustrated with cellular connectivity 2172 and wireless connectivity 2174. Cellular connectivity 2172 refers generally to cellular network connectivity provided by wireless carriers, such as provided via GSM (global system for mobile communications) or variations or derivatives, CDMA (code division multiple access) or variations or derivatives, TDM (time division multiplexing) or variations or derivatives, or other cellular service standards. Wireless connectivity (or wireless interface) 2174 refers to wireless connectivity that is not cellular, and can include personal area networks (such as Bluetooth, Near Field, etc.), local area networks (such as Wi-Fi), and/or wide area networks (such as WiMax), or other wireless communication.

Peripheral connections 2180 include hardware interfaces and connectors, as well as software components (e.g., drivers, protocol stacks) to make peripheral connections. It will be understood that the computing device 2100 could both be a peripheral device (“to” 2182) to other computing devices, as well as have peripheral devices (“from” 2184) connected to it. The computing device 2100 commonly has a “docking” connector to connect to other computing devices for purposes such as managing (e.g., downloading and/or uploading, changing, synchronizing) content on computing device 2100. Additionally, a docking connector can allow computing device 2100 to connect to certain peripherals that allow the computing device 2100 to control content output, for example, to audiovisual or other systems.

In addition to a proprietary docking connector or other proprietary connection hardware, the computing device 2100 can make peripheral connections 2180 via common or standards-based connectors. Common types can include a Universal Serial Bus (USB) connector (which can include any of a number of different hardware interfaces), DisplayPort including MiniDisplayPort (MDP), High Definition Multimedia Interface (HDMI), Firewire, or other types.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional element.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive

While the disclosure has been described in conjunction with specific embodiments thereof, many alternatives, modifications and variations of such embodiments will be apparent to those of ordinary skill in the art in light of the foregoing description. The embodiments of the disclosure are intended to embrace all such alternatives, modifications, and variations as to fall within the broad scope of the appended claims.

In addition, well known power/ground connections to integrated circuit (IC) chips and other components may or may not be shown within the presented figures, for simplicity of illustration and discussion, and so as not to obscure the disclosure. Further, arrangements may be shown in block diagram form in order to avoid obscuring the disclosure, and also in view of the fact that specifics with respect to implementation of such block diagram arrangements are highly dependent upon the platform within which the present disclosure is to be implemented (i.e., such specifics should be well within purview of one skilled in the art). Where specific details (e.g., circuits) are set forth in order to describe example embodiments of the disclosure, it should be apparent to one skilled in the art that the disclosure can be practiced without, or with variation of, these specific details. The description is thus to be regarded as illustrative instead of limiting.

The following examples pertain to further embodiments. Specifics in the examples may be used anywhere in one or more embodiments. All optional features of the apparatus described herein may also be implemented with respect to a method or process.

For example, an apparatus is provided which comprises: at least two sensors to sense a potential difference of a human body region; a computing device electrically coupled to the at least two sensors, wherein the computing device is to: detect a body hydration level according to the sensed potential difference; and notify a user if the body hydration level reaches a predetermined threshold. In some embodiments, the at least two sensors are to: inject a first current of a first frequency into the body region; and inject a second current of a second frequency in to the body region, wherein the first frequency is higher than the second frequency.

In some embodiments, the computing device is to: measure a first reactance using the first current of the first frequency; and measure a first resistance using the first current of the first frequency. In some embodiments, the computing device is to: measure a second reactance using the second current of the second frequency; and measure a second resistance using the second current of the first frequency. In some embodiments, the computing device is to: determine a phase angle according to the measured first reactance, first resistance, second reactance, and second resistance; and determine an indicator of human wellness according to the determined phase angle and anthropometric parameters.

In some embodiments, the anthropometric parameters include: age, gender, height, and weight. In some embodiments, the apparatus comprises a camera to capture a picture of a human, wherein the computing device is to analyze the captured picture to generate a user profile associated with the human body region. In some embodiments, the user profile comprises anthropometric parameters including: gender, age, and ethnicity. In some embodiments, the at least two sensors are part of: a bi-polar electrode system or a tetra-polar electrode system. In some embodiments, the bi-polar electrode system or the tetra-polar electrode system includes an electrode having an oleophilic coating. In some embodiments, the computing device is to notify the user at different time intervals about the body hydration level. In some embodiments, the predetermined threshold is near 2% of a baseline hydration level. In some embodiments, the one of more sensors are positioned: on keys of a keyboard, in a wearable device; or on a pad to be in contact with human palms. In some embodiments, the computing device is to: detect a body composition using the sensed potential difference; and correlate the body composition with cognitive efficiency using bio-impedance analysis.

In another example, a wearable device is provided which comprises: at least two sensors to sense a potential difference of a human body region; a processor electrically coupled to the at least two sensors, wherein the processor is to: detect a body hydration level according to the sensed potential difference; and notify a user if the body hydration level reaches a predetermined threshold; and a wireless interface for allowing the processor to communicate with another device. In some embodiments, the at least two sensors are part of: a bi-polar electrode system or a tetra-polar electrode system. In some embodiments, the bi-polar electrode system or the tetra-polar electrode system includes an electrode having an oleophilic coating.

In another example, a method is provided which comprises: receiving anthropometric parameters; sensing potential difference of a human body region; detecting a body hydration level according to the sensed potential difference; notifying a user if the body hydration level reaches a predetermined threshold; and determining an indicator of human wellness according to the sensed potential difference and the anthropometric parameters. In some embodiments, the method comprises: injecting a first current of a first frequency into the body region; injecting a second current of a second frequency in to the body region, wherein the first frequency is higher than the second frequency; measuring a first reactance using the first current of the first frequency; measuring a first resistance using the first current of the first frequency; measuring a second reactance using the second current of the second frequency; and measuring a second resistance using the second current of the first frequency. In some embodiments, the method comprises: determining a phase angle according to the measured first reactance, first resistance, second reactance, and second resistance, wherein determining the indicator of human wellness is also according to the determined phase angle.

In another example, an apparatus is provided which comprises: means for receiving anthropometric parameters; means for sensing potential difference of a human body region; means for detecting a body hydration level according to the sensed potential difference; means for notifying a user if the body hydration level reaches a predetermined threshold; and means for determining an indicator of human wellness according to the sensed potential difference and the anthropometric parameters. In some embodiments, the apparatus comprises means for injecting a first current of a first frequency into the body region; means for injecting a second current of a second frequency in to the body region, wherein the first frequency is higher than the second frequency; means for measuring a first reactance using the first current of the first frequency; means for measuring a first resistance using the first current of the first frequency; means for measuring a second reactance using the second current of the second frequency; and means for measuring a second resistance using the second current of the first frequency. In some embodiments, the apparatus comprises means for determining a phase angle according to the measured first reactance, first resistance, second reactance, and second resistance, wherein determining the indicator of human wellness is also according to the determined phase angle.

In another example, a wearable device is provided which comprises: at least two sensors to sense a potential difference of a human body region; a processor electrically coupled to the at least two sensors, wherein the processor comprises: means for detecting a body hydration level according to the sensed potential difference; and means for notifying a user if the body hydration level reaches a predetermined threshold; and a wireless interface for allowing the processor to communicate with another device. In some embodiments, the at least two sensors are part of: a bi-polar electrode system or a tetra-polar electrode system. In some embodiments, the bi-polar electrode system or the tetra-polar electrode system includes an electrode having an oleophilic coating.

An abstract is provided that will allow the reader to ascertain the nature and gist of the technical disclosure. The abstract is submitted with the understanding that it will not be used to limit the scope or meaning of the claims. The following claims are hereby incorporated into the detailed description, with each claim standing on its own as a separate embodiment.

We claim:

1. An apparatus comprising:
at least two sensors to sense a potential difference of a human body region;
a computing device electrically coupled to the at least two sensors, wherein the computing device is to:
detect a body hydration level according to the sensed potential difference; and
notify a user if the body hydration level reaches a predetermined threshold.

2. The apparatus of claim 1, wherein the at least two sensors are to:
inject a first current of a first frequency into the body region; and
inject a second current of a second frequency in to the body region, wherein the first frequency is higher than the second frequency.

3. The apparatus of claim 2, wherein the computing device is to:
measure a first reactance using the first current of the first frequency; and
measure a first resistance using the first current of the first frequency.

4. The apparatus of claim 3, wherein the computing device is to:
measure a second reactance using the second current of the second frequency; and
measure a second resistance using the second current of the first frequency.

5. The apparatus of claim 4, wherein the computing device is to:
determine a phase angle according to the measured first reactance, first resistance, second reactance, and second resistance; and
determine an indicator of human wellness according to the determined phase angle and anthropometric parameters.

6. The apparatus of claim 5, wherein the anthropometric parameters include: age, gender, height, and weight.

7. The apparatus of claim 1 comprises a camera to capture a picture of a human, wherein the computing device is to analyze the captured picture to generate a user profile associated with the human body region.

8. The apparatus of claim 7, wherein the user profile comprises anthropometric parameters including: gender, age, and ethnicity.

9. The apparatus of claim 1, wherein the at least two sensors are part of: a bi-polar electrode system or a tetra-polar electrode system.

10. The apparatus of claim 9, wherein the bi-polar electrode system or the tetra-polar electrode system includes an electrode having an oleophilic coating.

11. The apparatus of claim 1, wherein the computing device is to notify the user at different time intervals about the body hydration level.

12. The apparatus of claim 1, wherein the predetermined threshold is near 2% of a baseline hydration level.

13. The apparatus of claim 1, wherein the one of more sensors are positioned:
on keys of a keyboard;
in a wearable device; or
on a pad to be in contact with human palms.

14. The apparatus of claim 1, wherein the computing device is to:
detect a body composition using the sensed potential difference; and
correlate the body composition with cognitive efficiency using bio-impedance analysis.

15. A wearable device comprising:
at least two sensors to sense a potential difference of a human body region;
a processor electrically coupled to the at least two sensors, wherein the processor is to:
detect a body hydration level according to the sensed potential difference; and
notify a user if the body hydration level reaches a predetermined threshold; and
a wireless interface for allowing the processor to communicate with another device.

16. The wearable device of claim 15, wherein the at least two sensors are part of: a bi-polar electrode system or a tetra-polar electrode system.

17. The wearable device of claim 16, wherein the bi-polar electrode system or the tetra-polar electrode system includes an electrode having an oleophilic coating.

18. A method comprising:
receiving anthropometric parameters;
sensing potential difference of a human body region;
detecting a body hydration level according to the sensed potential difference;
notifying a user if the body hydration level reaches a predetermined threshold; and
determining an indicator of human wellness according to the sensed potential difference and the anthropometric parameters.

19. The method of claim 18 comprising:
injecting a first current of a first frequency into the body region;
injecting a second current of a second frequency in to the body region, wherein the first frequency is higher than the second frequency;
measuring a first reactance using the first current of the first frequency;
measuring a first resistance using the first current of the first frequency;
measuring a second reactance using the second current of the second frequency; and
measuring a second resistance using the second current of the first frequency.

20. The method of claim 19 comprising:
determining a phase angle according to the measured first reactance, first resistance, second reactance, and second resistance, wherein determining the indicator of human wellness is also according to the determined phase angle.
